(19) United States
(12) Patent Application Publication
(10) Pub. No.: US 2025/0295527 A1
(43) Pub. Date: Sep. 25, 2025

(54) IMPLANTABLE DEVICE FOR DEEP SCLERECTOMY

(71) Applicant: W. L. Gore & Associates, Inc., Newark, DE (US)

(72) Inventor: Kevin M. Savory, West Grove, PA (US)

(21) Appl. No.: 19/078,051

(22) Filed: Mar. 12, 2025

Related U.S. Application Data
(60) Provisional application No. 63/568,790, filed on Mar. 22, 2024.

(51) Int. Cl. A61F 9/007; A61F 2/00

(52) U.S. Cl. CPC A61F 9/00781; A61F 2/0077; A61F 2002/0081

(57) ABSTRACT

A scleral space implant for implantation in a scleral space includes a singular porous substrate having a top surface and an opposing bottom surface defining an implant interior and a consistent implant thickness disposed uniformly between the opposing top and bottom surfaces. The substrate has an implant rigidity that maintains the implant thickness when the scleral space implant is disposed in the scleral space. The top and bottom surfaces of the substrate each have a tissue-inhibiting pore structure that inhibits an ingress of ingrowing tissue into the implant interior while permitting the inflow of the aqueous humor into the implant interior. The implant interior has a coupled pore structure that facilitates a passage of the aqueous humor inflow through the implant interior.

### CROSS REFERENCE TO RELATED APPLICATIONS

The present application claims priority to U.S. Patent Application No. 63/568,790, filed Mar. 22, 2024, the entire disclosure of which is incorporated herein by reference.

### FIELD

The present disclosure relates generally to implantable devices. More specifically, the disclosure relates to medical devices that are used in deep sclerectomy.

### BACKGROUND

Glaucoma is a group of diseases that are frequently characterized by raised intraocular pressure (IOP). Glaucoma affects the optic nerve and may lead to blindness if not adequately treated. Patients with glaucoma may require surgical interventions to preserve their vision. When glaucoma continues to progress despite the use of medication regimes and possibly laser treatments, a glaucoma filtration procedure (trabeculectomy) may be recommended. Additional surgical techniques for reducing IOP include laser trabeculoplasty, non-penetrating filtration surgery, shunts, and cyclo-destructive procedures.

Deep sclerectomy (DS) is a non-penetrating filtration procedure that is used to surgically treat medically uncontrolled open-angle glaucoma, with less risk of complication as compared to trabeculectomy. In a DS procedure, the surgeon surgically removes a flap of deep sclera, deroofs Schlemm's canal, and remove the underlying juxtacanalicular trabecular meshwork, forming a scleral space through which aqueous humor would drain. By removing the juxtacanalicular meshwork, non-penetrating DS effectively targets the site of maximal outflow resistance, thereby increasing aqueous humor outflow and lowering IOP. By not penetrating full thickness through the trabecular meshwork into the anterior chamber, non-penetrating DS offers more reliability and safety than trabeculectomy.

However, non-penetrating DS has proven to be a technically difficult procedure with a long learning curve, and a high rate of conversion to trabeculectomy particularly in the hands of inexperienced surgeons. In some cases, a collagen implant, such as AquaFlow® (STAAR Surgical), is implanted in the scleral space formed during the DS procedure as explained above. The collagen implant may act as a stent to allow fluid to flow into the reservoir hidden below the ocular surface behind the scleral flap which now acts as a lid.

The collagen implant then dissolves after creating the new pathway for the excess IOP. However, because of the nature of collagen implants, the dissolving of the implant may inhibit the fluid flow through the new pathway over time due to tissue ingrowth causing an obstruction in the pathway. As such, there is a need for implantable medical devices that are designed to maintain the pathway to continuously allow fluid to flow from the anterior chamber in order to maintain the IOP within a preferred range for a prolonged duration of time.

### SUMMARY

In Example 1, a scleral space implant for implantation in a scleral space created during a deep sclerectomy surgical procedure performed on a human eye, the deep sclerectomy surgical procedure involving a removal of eye tissue to define the scleral space between a superficial scleral flap and a Schlemm's canal internal wall of the eye, the Schlemm's canal internal wall defining an inflow of an aqueous humor into the scleral space, the scleral space implant comprising a singular porous substrate having a top surface and an opposing bottom surface defining an implant interior and a consistent implant thickness disposed uniformly between the opposing top and bottom surfaces, the substrate further having an implant rigidity that maintains the implant thickness when the scleral space implant is disposed in the scleral space, wherein the top and bottom surfaces of the substrate each have a tissue-inhibiting pore structure that inhibits an ingress of ingrowing tissue into the implant interior while permitting the inflow of the aqueous humor into the implant interior, and wherein the implant interior has a coupled pore structure that facilitates a passage of the aqueous humor inflow through the implant interior.

In Example 2, the implant of Example 1, wherein the singular porous substrate further includes at least one exterior region having an ingrowth-permitting pore structure that permits the ingress of ingrowing tissue into the at least one exterior region.

In Example 3, the implant of Example 2, wherein the singular porous substrate has an open-tight-open-tight-open configuration.

In Example 4, the implant of at least one of Examples 1-3, wherein the top and bottom surfaces each define peripheral edges that mate to fully enclose the implant interior in at least one cross-sectional plane passing through the implant.

In Example 5, the implant of at least one of Examples 1-4, wherein the singular porous substrate is trimmable to define an implant profile conforming to the scleral space.

In Example 6, the implant of Example 5, wherein the implant, after being trimmed and implanted, is disposed entirely beneath the superficial scleral flap.

In Example 7, the implant of at least one of Examples 1-6, wherein the top surface defines a scleral flap plane for receiving the superficial scleral flap and maintaining the superficial scleral flap in a planar orientation.

In Example 8, the implant of at least one of Examples 1-7, wherein the passage extends from the bottom surface adjacent to the Schlemm's canal internal wall through the implant interior to an outflow surface of the implant facilitating a removal of the aqueous humor from the scleral space.

In Example 9, the implant of at least one of Examples 1-8, wherein the singular porous substrate includes a tail portion extending from the scleral space.

In Example 10, the implant of Example 9, wherein the outflow surface is disposed at the tail portion.

In Example 11, the implant of Example 10, wherein the tail portion is trimmable to be flush with an outer surface of the superficial scleral flap.

In Example 12, the implant of at least one of Examples 1-11, wherein the singular porous substrate includes polyethylene (PE), expanded polyethylene (ePE), expanded polytetrafluoroethylene (ePTFE), or silicone.

In Example 13, the implant of at least one of Examples 1-12, wherein the deep sclerectomy surgical procedure does not involve the use of an antimetabolite.

In Example 14, the implant of at least one of Examples 1-13, wherein the bottom surface includes a Schlemm's canal engagement portion disposed to receive the inflow of the aqueous humor into the scleral space.

In Example 15, the implant of at least one of Examples 1-14, wherein the tissue-inhibiting pore structure includes pores having a minimum pore size from 0.01 μm to 1 μm.

In Example 16, the implant of at least one of Examples 1-15, wherein the tissue-inhibiting pore structure includes pores having an average pore size from 0.01 μm to 1 μm.

In Example 17, the implant of at least one of Examples 1-16, wherein the coupled pore structure includes PE, ePE, ePTFE, or silicone.

In Example 18, the implant of at least one of Examples 1-17, wherein the coupled pore structure includes a first region with pores having a minimum pore size from 0.01 μm to 1 μm and a second region with pores having a maximum pore size from 1 μm to 100 μm.

In Example 19, the implant of at least one of Examples 1-18, wherein the coupled pore structure includes a first region with pores having an average pore size from 0.01 μm to 1 μm and a second region with pores having an average pore size from 1 μm to 100 μm.

In Example 20, the implant of at least one of Examples 1-19, wherein the ingrowth-permitting pore structure includes PE, ePE, ePTFE, or silicone.

In Example 21, the implant of at least one of Examples 1-20, wherein the ingrowth-permitting pore structure includes pores having a maximum pore size from 1 μm to 100 μm.

In Example 22, the implant of at least one of Examples 1-21, wherein the ingrowth-permitting pore structure includes pores having an average pore size from 1 μm to 100 μm.

In Example 23, a scleral space implant for implantation in a scleral space created during a deep sclerectomy surgical procedure performed on a human eye, the deep sclerectomy surgical procedure involving a removal of eye tissue to define the scleral space between a superficial scleral flap and a Schlemm's canal internal wall of the eye, the Schlemm's canal internal wall defining an inflow of an aqueous humor into the scleral space, the scleral space implant comprising a singular porous substrate having a top surface and an opposing bottom surface defining an implant interior and a consistent implant thickness disposed uniformly between the opposing top and bottom surfaces, the substrate further having an implant rigidity that maintains the implant thickness when the scleral space implant is disposed in the scleral space, wherein the singular porous substrate includes a tissue-inhibiting pore structure extending through a majority of the implant thickness, the tissue-inhibiting pore structure inhibiting an ingress of ingrowing tissue into the implant interior while permitting the inflow of the aqueous humor into the implant interior.

In Example 24, the implant of Example 23, wherein the tissue-inhibiting pore structure includes polyethylene (PE), expanded polyethylene (ePE), expanded polytetrafluoroethylene (ePTFE), or silicone.

In Example 25, the implant of at least one of Examples 23-24, wherein the top surface and the bottom surface have pores with a maximum pore size from 1 μm to 100 μm.

In Example 26, the implant of at least one of Examples 23-24, wherein the top surface and the bottom surface have pores with an average pore size from 1 μm to 100 μm.

In Example 27, the implant of at least one of Examples 23-26, wherein the bottom surface includes a Schlemm's canal engagement portion disposed to receive the inflow of the aqueous humor into the scleral space.

In Example 28, the implant of at least one of Examples 23-27, wherein the singular porous substrate is trimmable to define an implant profile conforming to the scleral space.

In Example 29, the implant of at least one of Examples 23-28, wherein the top surface defines a scleral flap plane for receiving the superficial scleral flap and maintaining the superficial scleral flap in a planar orientation.

In Example 30, the implant of at least one of Examples 23-29, wherein the singular porous substrate includes a tail portion extending from the scleral space.

In Example 31, the implant of at least one of Examples 23-30, wherein the deep sclerectomy surgical procedure does not involve the use of an antimetabolite.

In Example 32, the implant of at least one of Examples 23-31, wherein the deep sclerectomy surgical procedure maintains a natural healing capability of the tissue defining the scleral space.

In Example 33, a scleral space implant for implanting in a sclera of an eye to treat glaucoma in conjunction with a deep sclerectomy surgical procedure defining a scleral space disposed within surrounding tissues, the implant comprising a first external region having a first porosity that inhibits tissue ingrowth and facilitates flow of aqueous humor therethrough; a second external region having a second porosity that inhibits tissue ingrowth and facilitates flow of aqueous humor therethrough; and an internal region disposed between the first and second external regions, the internal region having a third porosity greater than the first and second porosities to facilitate fluid flow therethrough, wherein the first external region, the second external region, and the internal region have a stiffness to resist a contracting force applied from the surrounding tissues.

In Example 34, the implant of Example 33, further comprising an additional region disposed on the first external region such that the first external region is disposed between the additional region and the internal region, the additional region having a fourth porosity greater than the first and second porosities.

In Example 35, the implant of any one of Examples 33-34, wherein a portion of the first external region, the second external region, and the internal region defines a tail portion that extends from a remaining portion of the first external region, the second external region, and the internal region that defines a body portion, wherein the tail portion protrudes from a portion of the sclera in which the implant is implanted, such that only the body portion of the implant remains inside the sclera.

In Example 36, the implant of any one of Examples 33-35, wherein the first external region, the second external region, and the internal region include a plurality of nodes and a plurality of fibrils extending between the nodes to define pores between the fibrils.

In Example 37, the implant of any one of Examples 33-36, wherein the first external region includes a first partition, and the second external region includes a second partition, wherein the first partition and the second partition inhibit the tissue ingrowth into the internal region.

In Example 38, the implant of Example 37, wherein the internal region includes a plurality of support sections extending from the first partition to the second partition.

In Example 39, the implant of any one of Examples 33-38, wherein the implant is non-expanding and dimensionally stable when wetted.

In Example 40, the implant of any one of Examples 33-39, wherein the implant is made of one or more biocompatible and nonbiodegradable materials.

In Example 41, the implant of at least one of Examples 33-40, wherein the deep sclerectomy surgical procedure does not involve the use of an antimetabolite.

In Example 42, a method of draining excessive aqueous humor from a human eye to treat glaucoma in conjunction with a deep sclerectomy surgical procedure defining a scleral space, the method comprising directing an inflow from an exposed Schlemm's canal internal wall into an interior of a scleral space implant disposed in the scleral space; inhibiting an ingress of a tissue ingrowth from the scleral space into the implant interior; and channeling the inflow through the implant interior to an exit communicating with an exterior of the eye.

In Example 43, the method of Example 42, wherein the implant interior includes a coupled pore structure that facilitates the channeling of the inflow.

In Example 44, the method of any one of Examples 42-43, wherein the scleral space implant includes exterior surfaces having a tissue-inhibiting pore structure that inhibits the ingress of the tissue ingrowth into the implant interior.

In Example 45, the method of any one of Examples 42-44, further comprising trimming a peripheral edge of the scleral space implant to conform to the scleral space.

In Example 46, the method of any one of Examples 42-45, further comprising supporting a superficial scleral flap in a planar orientation.

In Example 47, the method of any one of Examples 42-46, wherein the tissue-inhibiting pore structure includes polyethylene (PE), expanded polyethylene (ePE), expanded polytetrafluoroethylene (ePTFE), or silicone.

In Example 48, the method of any one of Examples 42-47, wherein the coupled pore structure includes PE, ePE, ePTFE, or silicone.

In Example 49, the implant of at least one of Examples 42-48, wherein the deep sclerectomy surgical procedure does not involve the use of an antimetabolite.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 is a cross-sectional view of a portion of an eye in which an implantable device is implanted, according to embodiments disclosed herein.

FIG. 2A is an isometric view of an example of an implantable device according to embodiments disclosed herein.

FIG. 2B is an isometric view of another example of the implantable device according to embodiments disclosed herein.

FIG. 2C is an isometric view of an implantable device after being trimmed according to embodiments disclosed herein.

FIG. 2D is an isometric view of another example of an implantable device according to embodiments disclosed herein.

FIG. 2E is an isometric view of another example of an implantable device according to embodiments disclosed herein.

FIG. 2F is an isometric view of another example of an implantable device according to embodiments disclosed herein.

FIGS. 3A and 3B are partial views of a cross section of an eye during the procedure of implanting an implantable device according to embodiments disclosed herein.

FIGS. 4A, 4B, and 4C are partial views of a cross section of an eye during the procedure of implanting an implantable device according to embodiments disclosed herein.

FIG. 5 is an illustration showing possible fluid flow pathways within the implantable device according to embodiments disclosed herein.

FIG. 6 is a scanning electron microscopy (SEM) image of a cross sectional view of an implantable device according to embodiments disclosed herein (the image is to the scale shown in the image).

FIG. 7 is an SEM image of a surface of an implantable device according to embodiments disclosed herein (the image is to the scale shown in the image).

FIG. 8 is an SEM image of a cross sectional view of an implantable device according to embodiments disclosed herein (the image is to the scale shown in the image).

It should be understood that some of the drawings and replicas of the photographs may not necessarily be shown to scale, unless otherwise indicated. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### Description of Various Embodiments

The present disclosure relates to systems, devices, and methods for providing an implantable medical device for deep sclerectomy. As disclosed herein, the implantable medical device may achieve low inflammatory response and maintain an open fluid passage from the anterior chamber for reducing IOP for a prolonged duration of time without dissolving or facilitate tissue ingrowth through the entirety of the device.

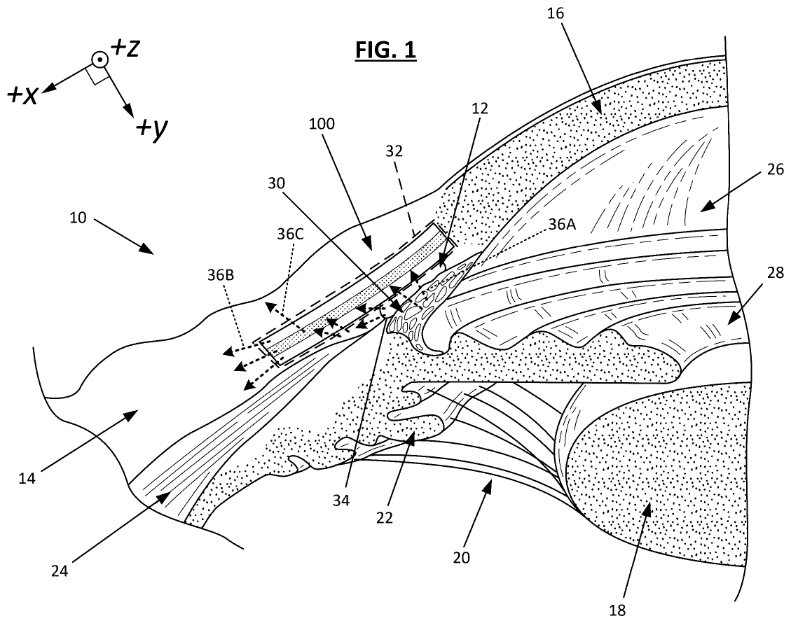

FIG. 1 shows a cross-sectional image of an eye 10 in which an implantable medical device 100 as disclosed herein may be implanted, according to examples disclosed herein. Hereinafter, the device 100 may be referred to as an implantable medical device or an implant. The figure shows a portion of the eye 10 surrounding a Schlemm's canal 12, the outer tissue layer of the eye includes a sclera 14, and in front of the sclera 14 is a cornea 16 that allows light to enter the interior of the eye. Inside the eye 10 is a lens 18 that is connected to the eye by zonular fibers 20 which are connected to the ciliary body 22.

Schlemm's canal 12 is a vascular structure in the eye 10 that has an internal wall 34 and functions to maintain fluid homeostasis by draining aqueous humor from the eye 10 into the systemic circulation. The ciliary muscle is made of smooth muscle fibers or ciliary muscle fibers 24 that are oriented in longitudinal, radial, and circular directions. Between the lens 18 and the cornea 16 is an anterior chamber 26 that contains aqueous humor.

Encircling the perimeter of the lens 18 is an iris 28 which forms a pupil around the approximate center of the lens 18. Light entering the eye 10 is optically focused through the cornea 16 and crystalline lens 18. The eye 10 also includes a trabecular meshwork 30 that is a fibrous network of tissue that encircles the iris 28 within the eye 10. The network of tissue layers that make up the trabecular meshwork 30 are porous and thus present a pathway for the egress of aqueous humor flowing from the anterior chamber 26.

As implanted, the device 100 occupies a surgically-created space 32 formed adjacent to the Schlemm's canal 12, between the trabecular meshwork 30 and the external layer of sclera 14 in the eye 10. The surgically-created space 32 is also referred to as a scleral space 32 created by a non-penetrating deep sclerectomy (DS) procedure, which is a surgical procedure that removes healthy sclera tissue near the Schlemm's canal to create a space to receive excess aqueous humor draining from the trabecular meshwork and the Schlemm's canal to therapeutically reduce IOP. The non-penetrating DS procedure may involve removal of the roof tissues of the Schlemm's canal 12, referred to as “deroofing”, to establish a new pathway for the drainage of the aqueous humor. Physicians performing the DS procedure anticipate the stimulation of a responsive healing process that may close the scleral space 32 over time. To inhibit the healing response and the subsequent reclosing of the scleral space, physicians may apply an antimetabolite to kill or inhibit surrounding tissue that would otherwise initiate or support the healing process. In some examples, the tissue-inhibiting (or ingrowth-inhibiting) pore structures of the implant or device 100 as disclosed herein can function to inhibit the refilling of the scleral space by tissue ingrowth which would allow the physician to avoid or limit the use of antimetabolites. In certain examples, the DS procedure that implements the implant or device 100 may be modified to avoid or limit the use of antimetabolites and retain or maintain a natural healing capability of the tissue defining the scleral space 32.

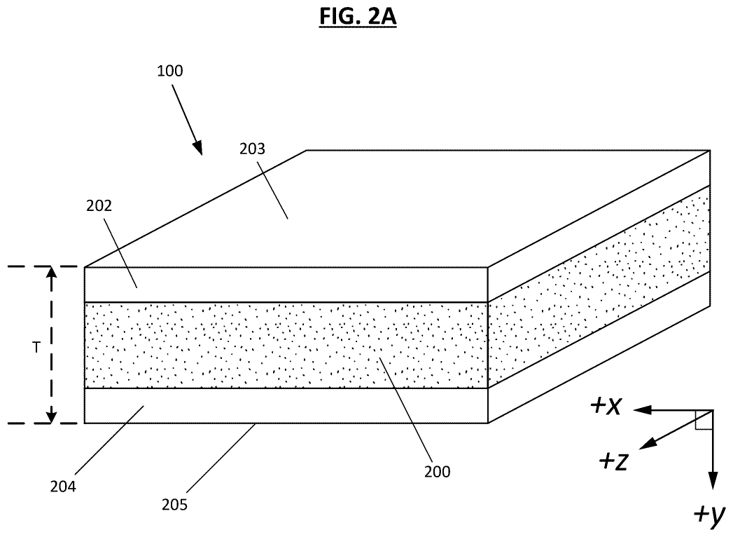

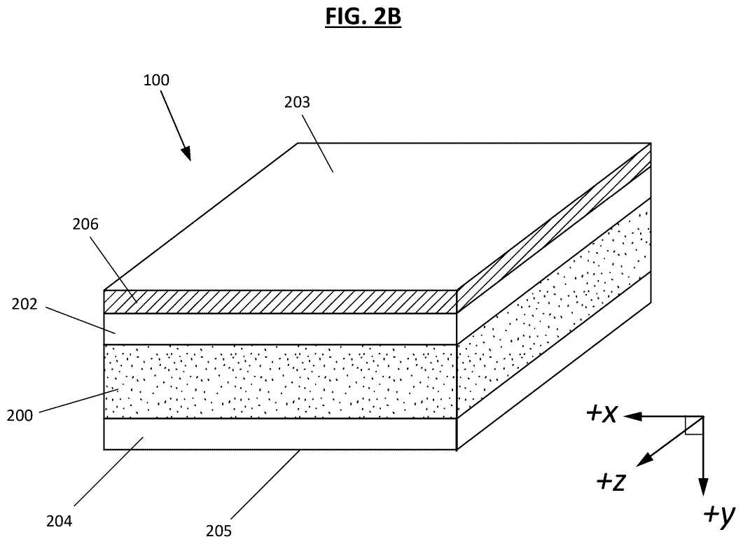

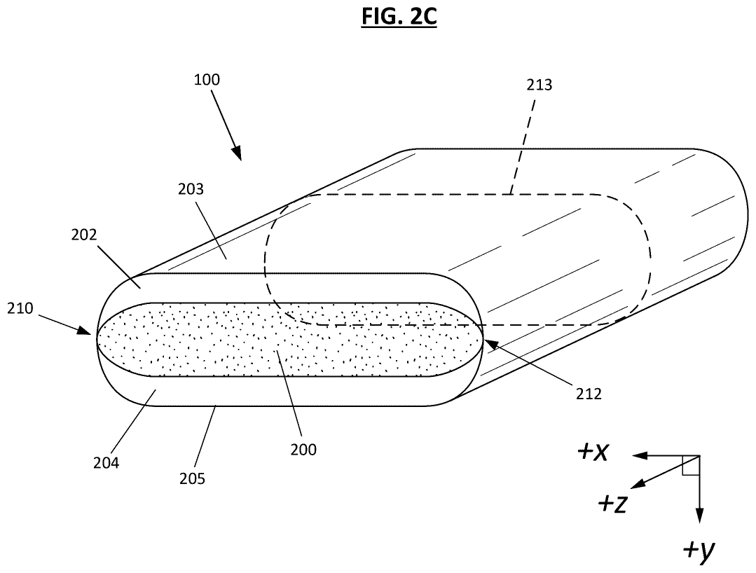

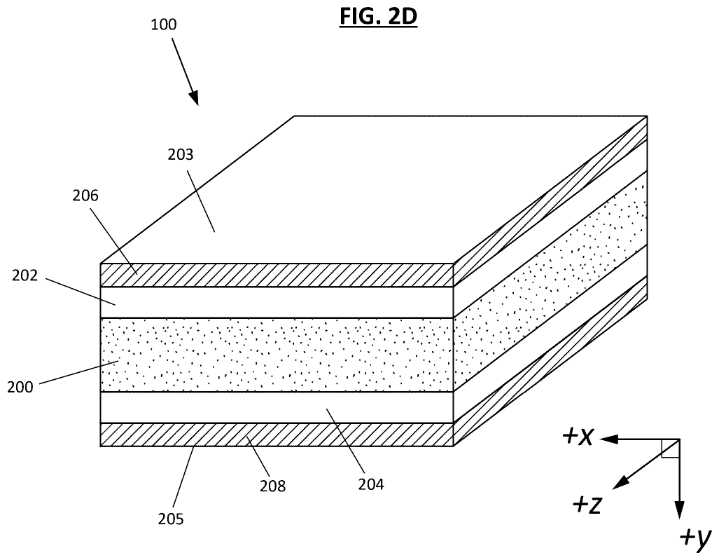

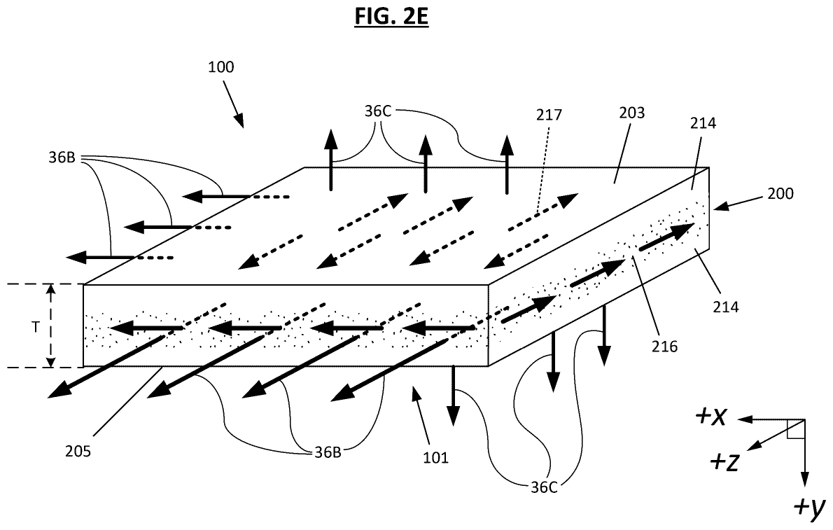

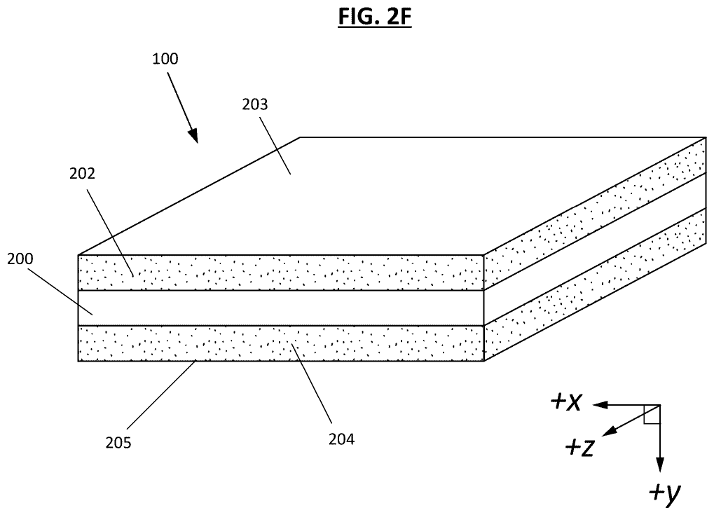

The device 100 may have a thickness (that is, the distance measured from the surface facing toward the Schlemm's canal 12 and the surface facing away from the Schlemm's canal 12) of no greater than 1 mm. The thickness is preferably consistent throughout the device 100 as illustrated in FIG. 2A and, in alternative designs, may have variations in thickness to accommodate anatomical structures or physician preference. In some examples, the thickness may be from 0.1 mm to 0.2 mm, from 0.2 mm to 0.5 mm, from 0.5 mm to 0.7 mm, from 0.7 mm to 1 mm, or any other value therebetween or combination of ranges thereof. In some examples, the device 100, when implanted, is nonexpanding and dimensionally stable when wetted. In some examples, the device 100 is made of one or more materials that are both biocompatible and nonbiodegradable. The internal wall 34 of the Schlemm's canal 12 defines an inflow 36A of an aqueous humor into the scleral space 32 such that the aqueous humor enters the Schlemm's canal 12 and then proceeds into the scleral space 32. As can be appreciated, the inflow 36A also represents a location where the aqueous humor enters the device 100. The implant or device 100 then directs the aqueous humor to the surrounding tissue in an outflow 36B from the device 100, which also represents a pathway by which the draining aqueous humor exits the scleral space at a distance from the Schlemm's canal. In some examples, the outflow 36B may be referred to as an exit 36B communicating with an exterior of the eye 10. As illustrated in FIG. 1, the outflow from the device 100 can also be from a side of the substrate at outflow 36C, which is similar to outflow 36B and similarly represents a pathway by which the draining aqueous humor exist the scleral space at a distance from the Schlemm's canal.

FIGS. 2A through 2F show isometric views of different examples or configurations of the device 100 according to embodiments disclosed herein. It is to be understood that, in each figure, the device 100 is shown such that the front-facing portion of the device 100 may substantially align with a plane defined by the x-axis and the y-axis as shown, and the device 100 may extend generally along the z-axis, where the x-axis, y-axis, and z-axis are perpendicular with respect to each other. In some examples, the positive x-direction may be defined as the direction away from the cornea 16 of the eye. In some examples, the positive y-direction may be defined as the direction toward the trabecular meshwork 30 of the eye, and/or the negative y-direction may be defined as the direction in which aqueous humor flows into the device from the trabecular meshwork 30. In some examples, the positive z-direction may be defined as the direction extending perpendicularly from both the positive x-direction and the positive y-direction.

FIG. 2A shows an example of the device 100 in which the device 100 has a substrate with three (3) layers: a first external layer 202, a second external layer 204, and an internal layer or implant interior 200 disposed between the external layers 202 and 204. As can be appreciated, the layers may present a clear boundary between adjacent layers as illustrated in FIGS. 2A and 2B or, alternatively, include irregular or undefined boundaries. The boundary between adjacent layers may, alternatively, be blended into each other or provide a gradual transition from one layer to the next. As can be further appreciated, the substrate may be formed by a joining of discrete layers that each have unique properties or, in a preferred embodiment, be formed from a material having properties that vary within the material and, in a more preferred embodiment, be formed from a material having properties that vary between the external surfaces of the material. The device 100 may assume a sufficiently planar configuration when positioned on a flat surface before being implanted. FIG. 2B shows an alternative embodiment with the device 100 having an additional layer 206 disposed external to the first external layer 202, such that the first external layer 202 is disposed between the additional layer 206 and the internal layer 200. In some examples not illustrated in FIG. 2B, the additional layer 206 may instead be disposed external to the second external layer 204 such that the second external layer 204 is disposed between the additional layer 206 and the internal layer 200, or may be disposed as a second additional layer 206 so that each side of the device 100 has an additional layer 206. Referring to FIG. 2A, the first external layer 202 defines a top surface 203, and the second external layer 202 defines an opposing bottom surface 205. As can be appreciated, embodiments having an additional layer or layers 206 may likewise be referred to as exterior layer(s) and define top surface 203 (as illustrated in FIG. 2B) and/or opposing bottom surface 205.

In some examples and as illustrated in FIGS. 2A-2D, the device 100 may have a thickness “T” that is a consistent implant thickness disposed uniformly between the top surface 203 and the bottom surface 205, where the device 100 has an implant rigidity that maintains its thickness when the device is implanted or disposed in the scleral space 32. The top surface 203 and the bottom surface 205 may each have a tissue-inhibiting structure, as further disclosed herein, that inhibits an ingress of ingrowing tissue into the internal layer 200 while permitting the inflow of the aqueous humor into the internal layer 200. The internal layer 200 may have a coupled pore structure, as further disclosed herein, that facilitates a passage of the aqueous humor inflow through the internal layer 200 of the device 100. For example, the passage may include any suitable fluid pathway or fluid passage between the inflow 36A and the outflow 36B, 36C (as shown in FIG. 1) and extending through the internal layer 200 in the direction of the flow of aqueous humor, as further disclosed herein. Furthermore, the additional layers 206 and 208 may have an ingrowth-permitting pore structure that permits the ingress of ingrowing tissue into the one or more additional layers 206 presented in some embodiments. It is believed that an ingress of ingrowing tissue into an exterior layer of the device may assist with anchoring the device within the scleral space with minimal interruption of a flow of aqueous humor into and through the internal layer.

The device 100 may be trimmable using any suitable tool such as a pair of surgical scissors and, as illustrated in FIG. 2C, the device 100 may present pinched or squeezed edges along the device periphery that may cause a reduction in the device thickness at the portions that are cut. The pinched or squeezed edges may be presented after the device is trimmed with a cut using the scissors and, alternatively, the pinched or squeezed edges may be preformed into the device prior to a trimming procedure. For example, in FIG. 2C, a first side edge portion 210 and a second side edge portion 212 may be trimmed to conform to the scleral space or be reduced in thickness as compared to the rest of the device 100, measured from an external surface of the first external layer 202 and an external surface of the second external layer 204. The edge portions 210 and 212 may also be referred to as peripheral edges. For example, the top surface 203 and the bottom surface 205 may each include a plurality of peripheral edges 210, 212 that each mates with a corresponding peripheral edge from the opposing surface. Doing so causes the interior of the implant, for example the internal layer 200, to be fully enclosed by external layers 202 and 204 in at least one cross-sectional plane 213 passing through the device 100. As shown, the cross-sectional plane 213 is a perpendicular cross-sectional plane that intersects the device 100 along the x and y directions, although any other suitable cross-sectional plane may be implemented. As can be appreciated, the full enclosure of the sides of the substrate with the external layers 202 and 204 may inhibit aqueous humor drainage from the internal layer 200 to the side outflows 36C illustrated in and described with regard to FIGS. 1, 2E and 3B, thereby leaving only outflow 36B as the path for aqueous humor passing through internal layer 200 and exiting from the implant 100 into the scleral space.

The first external layer 202, the second external layer 204, and the internal layer 200 have a sufficient stiffness to resist in vivo a contracting force applied from surrounding tissue when the device 100 is implanted. The contracting force may be caused by scar tissue that is formed around the device 100. For example, after the non-penetrating DS procedure is performed, deposits of collagen may form inside the scleral space in which the device 100 is implanted. Over time, the collagen contracts to densify, forming an abnormal deposit of dense collagen in and surrounding the portion of the eye in which the implant 100 is disposed. The increased density of the collagen is believed to reduce the flow of aqueous humor through the scleral space. Therefore, a stiffness sufficient to resist the contracting force applied by such densifying collagen in the scarred tissues is desirable as it helps maintain consistent and reliable fluid flow for a prolonged period of time after the device 100 is implanted.

In some examples and with reference to FIG. 2A, the external layers 202 and 204 have sufficiently small pore sizes to inhibit tissue ingrowth therethrough. For example, the characteristics of a layer that facilitates tissue ingrowth may include a greater average pore size, such as pore sizes that are greater than a typical size of a cell forming the potentially-ingrowing tissue that surroundings the device when implanted in the scleral space, as further explained herein. Furthermore, in contrast, the external layers 202 and 204 may prevent, inhibit, or discourage tissue ingrowth into the internal layer 200 by having one or more regions or sections in the external layers 202 and 204 that have a sufficiently small average pore size to inhibit tissue cells from significantly penetrating the layers while maintaining flow of liquid therethrough, where the liquid passing into the internal layer 200 may include aqueous humor from the anterior chamber 26 of the eye 10, for example. In some examples, the inclusion of an additional layer 206 may have greater average pore size that facilitates cell penetration to form tissue ingrowth by having a greater average pore size than the average pore size of the external layer 202 on top of which the additional layer 206 is disposed.

FIG. 2D shows another example of the device 100 in which the device 100 has a substate with five (5) layers: the first external layer 202, the second external layer 204, the internal layer 200 disposed between the external layers 202 and 204, a first additional layer 206, and a second additional layer 208. The first additional layer 206 is disposed on the first external layer 202 such that the first external layer 202 is disposed between the first additional layer 206 and the internal layer 200, as discussed with regard to FIG. 2C. The second additional layer 208 may be disposed on the second external layer 204 such that the second external layer 204 is disposed between the second additional layer 208 and the internal layer 200. The additional layer(s) 206 and/or 208 may have an average pore size that is greater than the average pore size of the external layer(s) 202 and/or 204. In this example of FIG. 2D, the additional layer 206 has an average pore size that allows tissue ingrowth and defines the top surface 203, and the additional layer 208 has an average pore that allows tissue ingrowth and defines the opposing bottom surface 205. In some examples, the average pore size of the additional layer(s) 206, 208 may have the same average pore size present in the internal layer 200. In some examples, the structure of the additional layer(s) may be substantially similar to the structure of the internal layer 200, which may include a distribution of the pores in the material, a quantity of pores in the material, a depth of the pores in the material, or a cross-connectivity of adjacent pores within the material.

FIG. 2E shows another example of the device 100 in which the device 100 has a singular substrate 101 having the top surface 203 and the opposing bottom surface 205 defining the implant interior 200 disposed therebetween. The substrate 101 has a consistent implant thickness “T” disposed uniformly between the opposing top and bottom surfaces, and the substrate 101 further has an implant rigidity that maintains the implant thickness when the device 100 is disposed in the scleral space 32. In some examples, the substrate 101 may be referred to as a unitary substrate or a monolithic substrate, due to the substrate being made of a single continuous piece of material such that the substrate is not formed by attaching together two or more pieces of material.

Alternatively, the substrate 101 may be formed from two or more discrete layers joined together to provide a singular structure. In some embodiments, the substrate 101 can possess material characteristics, such as average pore size, that is consistent throughout the substrate. As can be appreciated, additional material characteristics may be consistent throughout the substrate, such as a distribution of pores in the material, a quantity of pores in the material, a depth of the pores in the material, or a cross-connectivity of adjacent pores within the material. Also shown in FIG. 2E are a plurality of arrows, where the solid bold arrows indicate the flow of fluid from inside the device 100 to outside (or exterior) of the device 100, and the dotted arrows indicate the flow of fluid within the device 100. Within the device 100, fluid may flow in any direction generally along the x-z plane as defined by the x-axis and the z-axis.

In some examples, the porous substrate 101 may have an implant rigidity that maintains the implant thickness when the device 100 or the substrate 101 is disposed in the scleral space 32, and also the substrate 101 may include a tissue-inhibiting pore structure 214 extending through a majority of the thickness “T” of the substrate 101, from each surface 203, 205 towards the implant interior 200 or from only a single surface of the substrate. The majority of the thickness may refer to percentage of a total thickness of the material possessing the selected material characteristic, and may be expressed as a percentage greater than 50%, and in some examples, the percentage may be at least 60%, at least 70%, at least 80%, or at least 90% of the entire thickness of the substrate 101, or any other suitable range therebetween. That is, the implant interior 200 may be formed primarily of the tissue-inhibiting pore structure 214 without the interconnected coupled pore structure 216 that facilitates passage 217 of the aqueous humor inflow through the implant interior 200 as illustrated in the embodiment of FIG. 2E (e.g., the dotted arrows). In such a configuration, the entirety of the substrate 101 may resemble the external layer 202 or 204 as shown in FIG. 2A. Alternatively, in some examples, the substrate 101 may additionally resemble the external layer 202 or 204 of the embodiment illustrated in FIG. 2A but also include the coupled pore structure 216 so as to facilitate the passage 217 of aqueous humor inflow through the implant interior 200, as shown in FIG. 2E, where the substrate 101 has two regions or sections (upper and lower) having the tissue-inhibiting pore structure 214 defining both the top surface 203 and the bottom surface 205, while maintaining the coupled pore structure 216 between the two regions or sections of the tissue-inhibiting pore structure 214.

Also illustrated in FIG. 2E are the outflows 36B, 36C showing how the aqueous humor travelling the passage 217 may exit the device 100 at a terminal end near outflow 36B (which may be directed along a positive x-direction and/or a positive/negative z-direction) or at the sides of the device at outflows 36C (which may be directed along a positive/negative y-direction). The outflows 36C may be outflows from external sides of the device, such as from the top surface 203 and/or the bottom surface 205 of the device 100. As can be appreciated, the passage 217 defines a pathway that leads from the inflow 36A (not shown in FIG. 2E) to the outflows 36B, 36C because of a pressure gradient defined by the inflow of aqueous humor into the scleral space near the bottom surface 205, as described above.

FIG. 2F shows an example of the device 100 in which the device 100 has a substrate with three (3) layers: a first external layer 202, a second external layer 204, and an internal layer or implant interior 200 disposed between the external layers 202 and 204. In this example, the internal layer or implant interior 200 is composed of a material or substrate that includes a tissue-inhibiting pore structure, whereas the external layers 202 and 204 are composed of a material or substrate that includes an ingrowth-permitting pore structure, or a coupled pore structure. Therefore, the top surface 203 and the bottom surface 205 may at least partially include ingrowth-permitting pore structures, or coupled pore structures.

In some examples, any portion of the device 100 (e.g., any one or more of the layers) may include one or more biocompatible materials such as expanded polytetrafluoroethylene (ePTFE). Additionally, the one or more component(s) of the device 100 may be formed of other biocompatible materials including biocompatible polymers, which may or may not be microporous, including, but not limited to, polyurethane (PU), silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, expanded polyethylene (ePE), and polytetrafluoroethylene (PTFE). In some examples, the materials may include electrospun polyurethane.

In some examples, materials forming the tissue-inhibiting pore structures may include polyethylene (PE), ePE, ePTFE, and/or silicone. In some examples, materials forming the coupled pore structures may include PE, ePE, ePTFE, and/or silicone. In some examples, materials forming the ingrowth-permitting pore structures may include PE, ePE, ePTFE, and/or silicone. In some examples, certain materials such as PE, PU, polyamide-based polymers, silicone, etc., may be used to form a plurality of fused structures such as fused beads, in order to create pores. Such fused structure may be formed via sintering, for example, or any suitable methods in which particulates within the material are compressed or heated/chemically reacted to fuse, thereby increase density within the material. In some examples, doing so may facilitate maintaining channels within structures of the material so as to allow passage of fluid (such as aqueous humor) therethrough. In some examples, the tissue-inhibiting pore structures, coupled pore structures, and ingrowth-permitting pore structures may be formed using the same material (or combination of materials) but processed using different methods (including but not limited to sintering and electrospinning, for example) so as to achieve the different properties (e.g., average, maximum, or minimum pore sizes) as described herein.

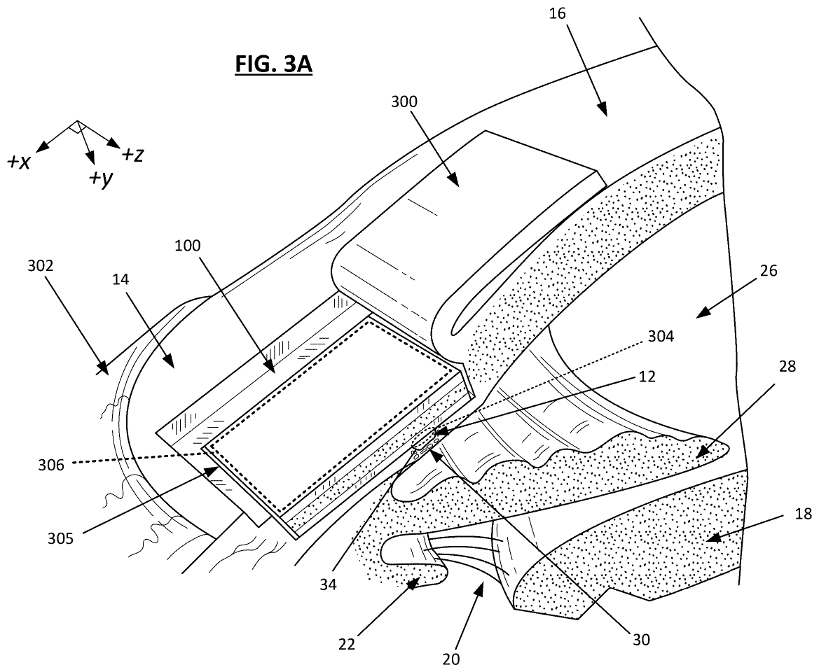

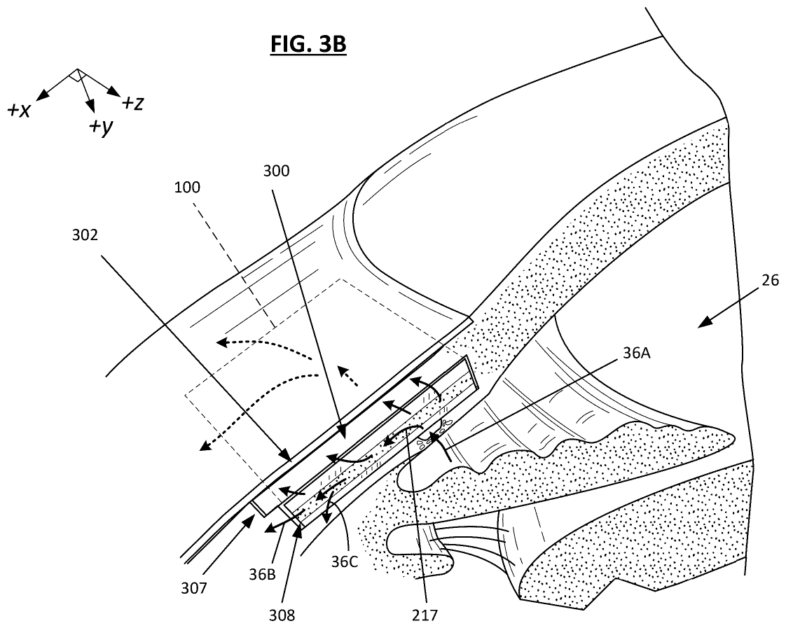

FIG. 3A shows a portion of the eye during the procedure of non-penetrating DS, in which conjunctival tissue 302 has been partially pulled back and a portion of the scleral tissue, or sclera 14, has been cut and pulled back (in a direction opposite from the direction in which the conjunctival tissue 302 is pulled back). The pulled-back portion of the scleral tissue 14 is hereinafter referred to a “flap” 300. At this point in the DS procedure the Schlemm's canal 12 has been deroofed. A portion of the sclera 14 that was located underneath the flap 300 has been cut and removed and the scleral space has been prepared to receive aqueous humor exiting the Schlemm's canal. After the scleral space is prepared, the device 100 is shown disposed inside the scleral space to occupy the volume that was previous occupied by the excised tissue underneath the flap 300. As illustrated in FIG. 3B, after implanting the device 100, the DS procedure is completed when the flap 300 is returned to its original position, and the conjunctival tissue 302 is pulled toward the cornea 16, thereafter covering the flap 300. As such, the device 100 is disposed entirely beneath the flap 300 and the conjunctival tissue 302. As implanted, the device 100 may have the second external layer 204 facing and defining a portion of the Schlemm's canal 12.

In some examples, the device 100, referred to herein as a scleral space implant, is implanted in the scleral space 32 that is created during a DS surgical procedure performed on the human eye 10. The DS surgical procedure involves removing healthy eye tissue to define the scleral space 32 between the superficial scleral flap 300 and an internal wall 34 of the Schlemm's canal 12 of the eye 10, also referred to as the Schlemm's canal internal wall 34. The Schlemm's canal internal wall 34 defines an inflow 36A of an aqueous humor into the scleral space 32 such that the aqueous humor enters the Schlemm's canal 12 and then into the scleral space 32, subsequently entering the device 100 at the inflow 36A where in the inflow 36A passing into the bottom surface 205 of the device 100. The implant or device 100 then directs the aqueous humor to the surrounding tissue towards and via an outflow 36B and, in some embodiments, an outflow 36C.

In some examples, the bottom surface 205 of the implant 100 has an engagement portion 304 for the Schlemm's canal 12, also referred to as the Schlemm's canal engagement portion 304. The engagement portion 304 is a portion of the bottom surface 205 that is defined and disposed to receive the inflow 36A of aqueous humor into the scleral space 32. The engagement portion 304 may also act or substitute as the “roof” of the Schlemm's canal 12 due to the canal having been deroofed as a result of the DS procedure, as explained above. The implant 100 may also be trimmable to define an implant profile 305 that conforms to the scleral space 32.

In some examples, the top surface 203 of the device 100 defines a scleral flap plane 306, which is the plane configured to receive the superficial scleral flap 300 and may support or maintain the flap 300 in a planar orientation 307 that is similar to an original position of the flap tissue prior to the DS procedure, as shown in FIG. 3B. It is believed that the flap 300 and the surrounding tissue will facilitate the outflow of the aqueous humor exiting the device 100 to allow for drainage and a therapeutic reduction in IOP. In some examples, the passage 217 may extend from the bottom surface 205 adjacent to the Schlemm's canal internal wall 34 through the implant interior 200 to an outflow surface 308 of the implant 100 to facilitate a removal of the aqueous humor from the scleral space 32.

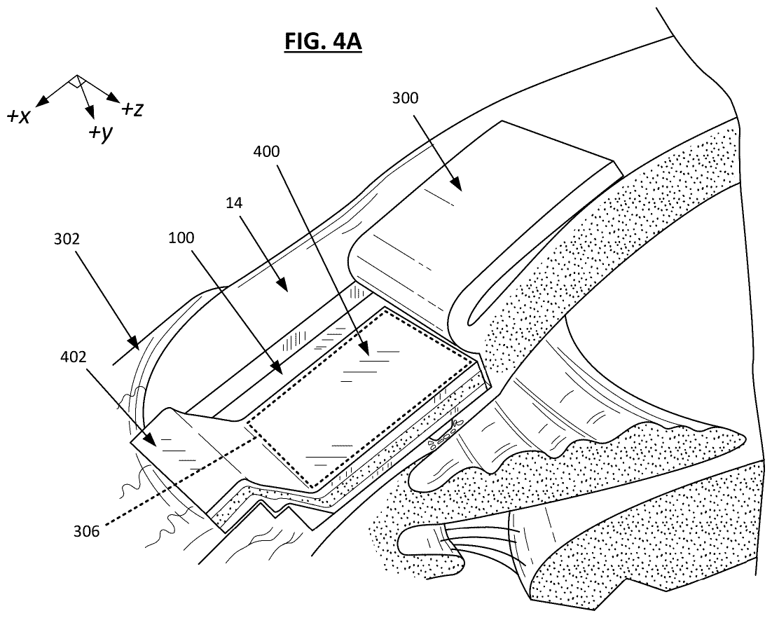

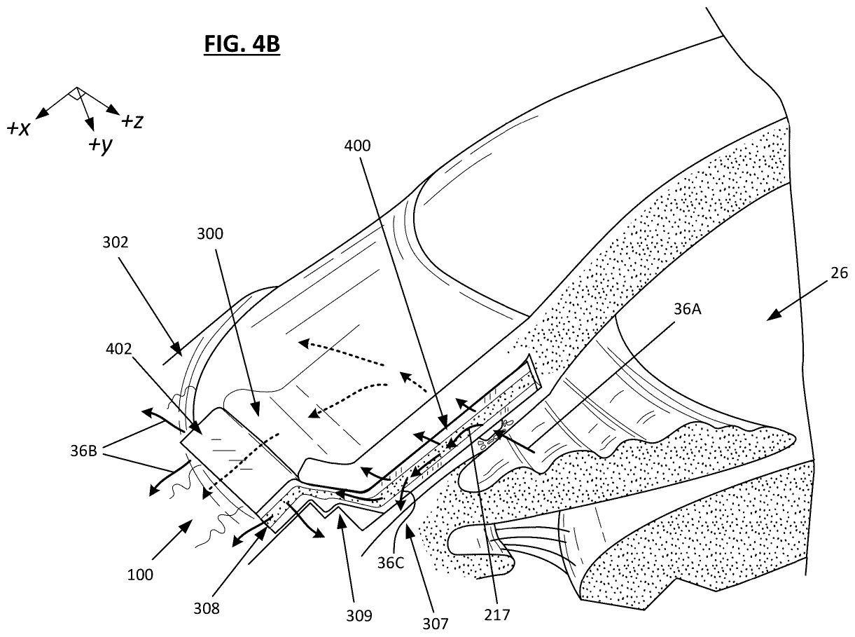

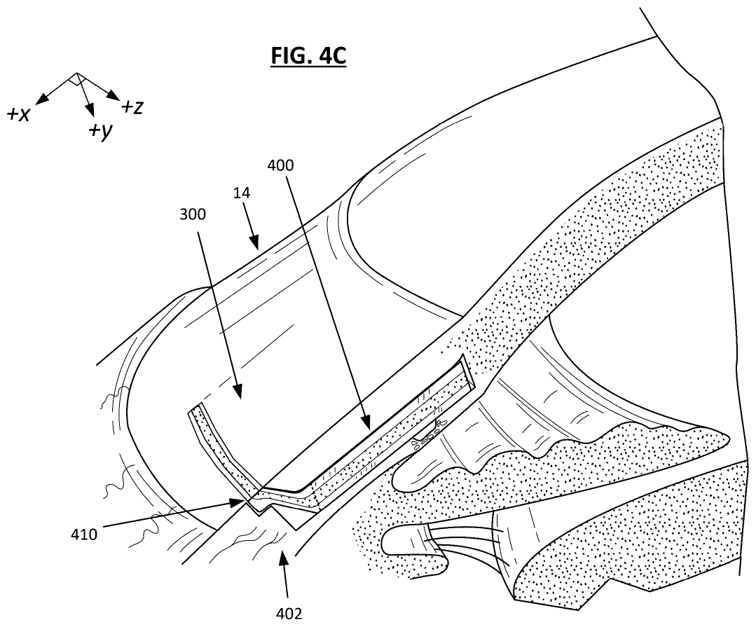

FIG. 4A shows another example of the device 100 in which a tail portion 402 of the device 100 protrudes from the cut portion of the sclera 14, such that only a body portion 400 of the device 100 remains inside the cut portion and underneath the flap 300 as shown in FIG. 4B. In this example, the tail portion 402 would remain exposed and not covered by the flap 300 to allow drainage of aqueous humor directly to a space external to the eye without passing through the repositioned scleral flap 300. The tail portion 402 also includes a portion of the external layers 202 and 204 as well as a portion of the internal layer 200. In some examples, the tail portion 402 acts as a passageway for the fluid (e.g., aqueous humor) to pass into the space between the conjunctival tissue 302 and the scleral tissue 14, thereby helping to reduce or eliminate the increase of IOP due to the buildup of fluid inside the anterior chamber 26. In some examples, the outflow surface 308 may be disposed at the tail portion 402. In some embodiments the external layers 202, 204 are absent where the device extends away from the scleral space and the drainage of the aqueous humor is via a fully-exposed portion of the internal layer 200 communicating with a space external to the eye.

In some examples, a portion of the top surface 203 defines the scleral flap plane 306, which is the plane configured to receive the superficial scleral flap 300 and maintains a portion of the flap 300 in the planar orientation 307. The portion of the flap 300 that is not maintained in the planar orientation 307 (that is, the portion of the flap 300 that does not come into contact with the portion of the top surface 203 of the device 100 that defines the scleral flap plane 306) may be raised upward with respect to the scleral flap plane 306, thereby maintained in a raised orientation 309. Both the planar orientation 307 and the raised orientation 309 assumed by the aforementioned portions of the flap 300 are shown in FIG. 4B.

In other examples, the scleral flap 300 may be trimmed or positioned to prevent, inhibit, or limit the formation of a raised orientation 309. In further embodiments, the tail portion 402 may be shortened so that its terminal end coincides with the planar surface defined by the scleral flap plane 306 so as to provide direct drainage of aqueous humor (without or in addition to passage of aqueous humor through the scleral flap 300) while presenting an uninterrupted and/or a mostly natural exterior surface of the sclera. FIG. 4C shows an example of the device as implanted, where the scleral flap 300 is trimmed to prevent, inhibit, or limit the formation of the raised orientation 309, and the tail portion 402 of the device 100 is shortened by trimming or cutting off a portion of the tail portion 402 such that an end 410 of the tail portion 402 is substantially flush with an outer surface of the scleral flap 300 and with a surface of the scleral tissue 14 of the eye.

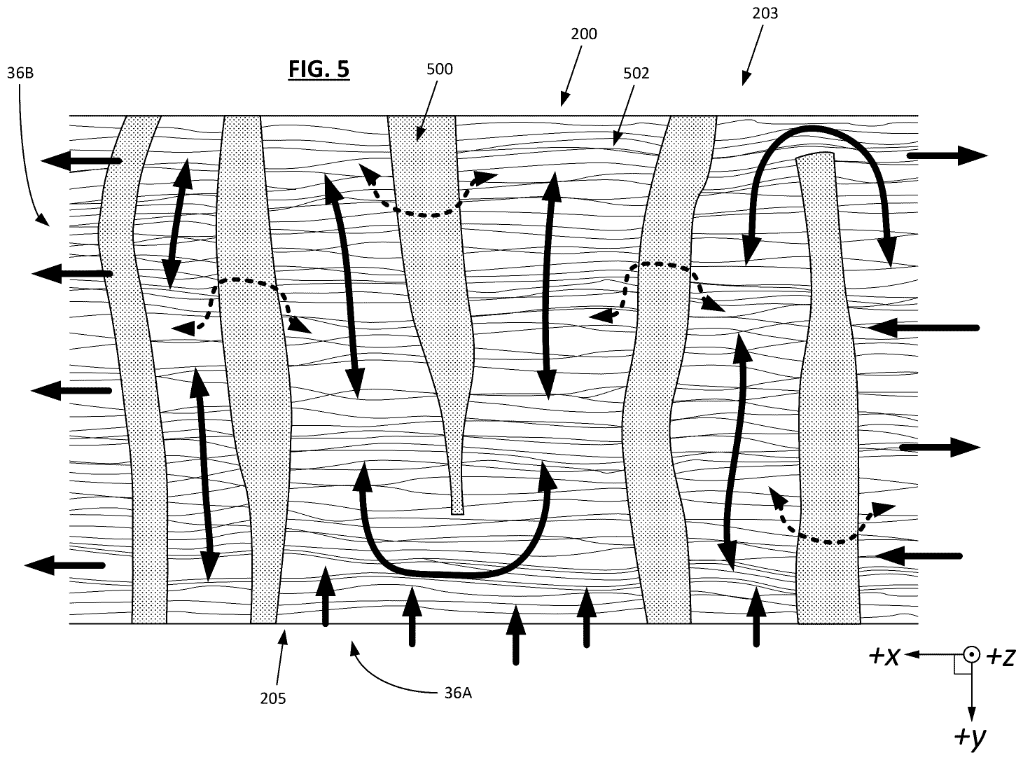

In some embodiments, the pore structure may be defined by a material that defines pores with an expanded polymer material. FIG. 5 shows an expanded polymer material having a plurality of nodes 500 and fibrils 502 which may occupy the space inside the internal region 200 according to embodiments disclosed herein. A node may be any section having a “clump” or larger volume of polymer when compared to a fibril. The arrows indicate potential directions in which fluid may flow within the internal region 200 to define a coupled pore structure where one pore communicates to one or more adjacent pores to allow fluid communication in a direction that is in part parallel to the exterior surfaces of the material. The bold arrows indicate the directions of potential fluid flow along a plane defined by the x-axis and the y-axis as shown on the bottom right-hand corner of FIG. 5, whereas the dotted arrows indicate the directions that protrude out of the page (i.e., out of the plane defined by the x-axis and the y-axis) or into the page, therefore indicating a flow that extends at least partially along the z-axis as shown, in order to circumvent the nodes. As can be appreciated, the coupled flow structure illustrated in FIG. 5 presents internal flow pathways that permit aqueous humor migration in multiple directions within the internal region 200 and, as can be further appreciated, the presence of a pressure gradient in that aqueous humor flow can drive the aqueous humor from an inflow 36A position to an outflow 36B position of the device 100 and, in some embodiments, to an outflow 36C position of the device 100. Also, as can be appreciated, the direction of flow is preferential in the direction of least resistance, such as in view of fluid transport as measured or observed in vivo.

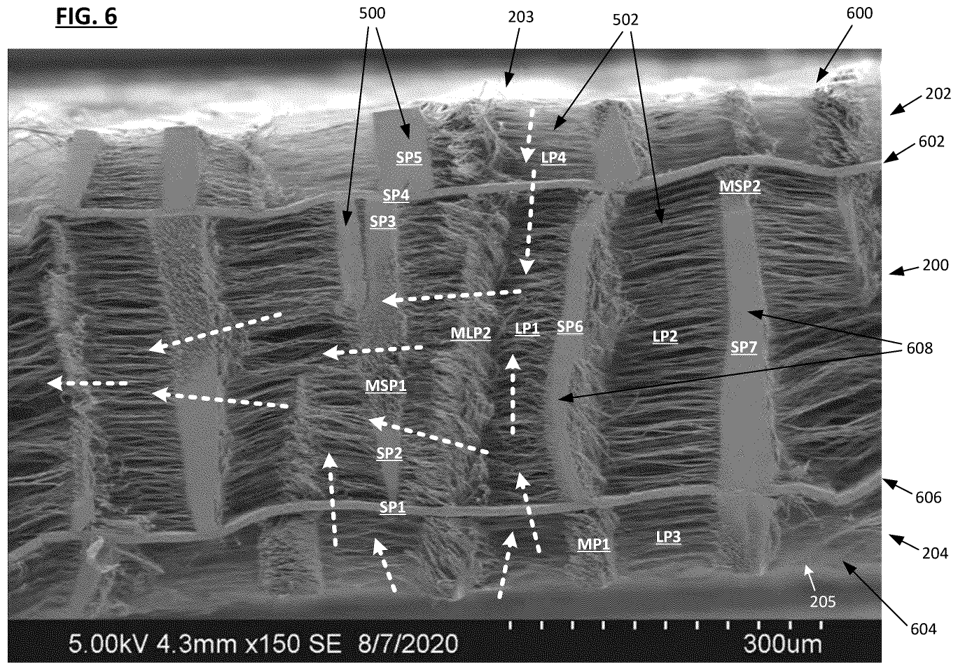

FIG. 6 shows a microscopic view of a microporous material of the implantable device 100 according to some embodiments. Displayed at the bottom of FIG. 6 is: “5.00 kV 4.3 mm×150 SE 8/7/2020” and a scale with the distance between two consecutive lines of the scale, as shown at the bottom right hand corner, representing 30 μm. For example, the microporous material of FIG. 6 may be referred to throughout with reference to an implantable device or system and present an embodiment that provides at different locations within the same material pores and pore structures that are (1) tissue-inhibiting (ingrowth-inhibiting) pore structures, (2) coupled pore structures, and (3) ingrowth-permitting pore structures, as explained further below. As can be appreciated by a person of skill in the art and with reference to FIG. 6, the microporous aspects and parameters of the microporous material can be defined in a variety of ways. In an application of a microporous material in an ocular device, such as the implantable device 100 described herein, configured for in situ placement in the tissue of the eye to control an internal fluid pressure of the eye (IOP), the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or another fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in the device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate pressure control in the eye for the treatment of a disease.

For simplicity, the aforementioned characteristics and variables of the microporous material used in the various embodiments and examples described herein can be presented simply as a porosity which can be based upon a volumetric porosity, a pore or passage size, or a tortuosity metric. Again, with reference to FIG. 6, internal portions of the microporous material can have varying porosities (or volumetric porosities, or pore sizes, or tortuosities).

In some examples, the device 100 may include two regions of barrier or partition 602 and 606 that separate the device 100 into the external portions (e.g., 202 and 204) and the internal portion (e.g., 200). The barriers or partitions may be located horizontally. In some examples, the first barrier or partition region 602 may be included in the first external region 202, and the second barrier or partition region 606 may be included in the second external region 204. In some examples, the first barrier or partition 602 may define the position of the pores in the first external region 202 due to the length and position of the nodes 500 extending from the first barrier or partition 602 into the first external region 202, and the second barrier or partition 606 may define the position of the pores in the second external region 204 due to the length and position of the nodes 500 extending from the second barrier or partition 606 into the second external region 204. In some examples, the internal region 200 may include a plurality of support sections 608 extending between the partitions 602 and 606. The support sections 608 provide resistance for the internal region 200 against external forces applied to the internal region 200 (or more generally, to the device 100 itself). In some examples, the support sections 608 may extend vertically from the first partition 602 to the second partition 606. In some examples, one of the support sections 608 may extend only partially between the partitions 602 and 606, but when combined with a neighboring support section, the two or more support sections 608 may extend the entire distance between the partitions 602 and 606, and since the two support sections are nodes 500 that are interconnected via a plurality of fibrils 502, the two support sections may work together to provide support for the device 100 against external forces being applied thereto.

At any of these portions of the device 100, the porosity can comparatively range in degree from small pore size (SP), medium-small pore size (MSP), medium pore size (MP), medium-large pore size (MLP), and large pore size (LP), where LP is larger than MLP, MLP is larger than MP, MP is larger than MSP, and MSP is larger than SP. In some examples, the size of SP may range from about 0.01% to 1%, MSP may range from about 1% to 5%, MP may range from about 5% to 20%, and MLP may range from about 20% to 80% that of the size of LP. The size of SP may range from about 0.01 μm to about 1 μm in pore diameter (or pore average dimension). In some examples, the porosity may increase by about 5 to 10 times as the pore size increases from one category to the next category (for example, from SP to MSP or from MSP to MP, etc.).

In some embodiments, the device 100 is essentially a monolithic, unitary member. The physical properties of the barriers or partitions 602 and 606, the external portions 202 and 204 and the internal portion 200 have some similarities and some differences. As stated previously, one similarity is that they are all fluid-permeable, meaning that aqueous humor is able to traverse through the device 100. One difference is that the barriers or partitions 602 and 606 have a “tight” configuration that is cell-impermeable while the portions 200, 202, and 204 have an “open” configuration. The open configuration is cell-permeable, meaning that tissue ingrowth is permitted by the open configuration of the external portions 202 and 204 but prohibited by the tight configuration of the barriers or partitions 602 and 606. Furthermore, while the barriers or partitions 602 and 606 allow the passage of the aqueous humor, the open configuration and coupled pores of the internal portion 200 facilitates the flow of the aqueous humor through the device 100. In other words, the flow resistance through the internal portion 200 is significantly less than the flow resistance through the barriers or partitions 602 and 606.

With reference to the aforementioned embodiments, the aqueous humor drainage may travel along a relatively straight path through a microporous material so as to sequentially engage porosities of a second surface 205 of the second external portion 204 and then enter a uniform internal portion 200 and, in a similar manner, drainage from another direction may travel through a first surface 203 of the first external portion 202 and then enter the internal portion 200. For instance, the partitions 602 and 606 may have a low porosity throughout (e.g., to resist tissue ingrowth into the internal portion 200, and portions of the interior portions (e.g., 200) and the external surfaces (e.g., 600 and 604) can have any of the aforementioned degrees of porosity. Under these circumstances, fluid may be delivered through the microporous material from a region with low porosity (small pore size SP) to a region with high porosity (large pore LP) while passing through a region with medium porosity (medium pore size MP) can be represented as SP-MP-LP. More examples are discussed here below. This arrangement of pore structures and porosities can exemplify an ingrowth-permitting pore structure at external portions 202 and 204, exemplify a tissue-inhibiting (or ingrowth-inhibiting) pore structure at partitions 602 and 606, and exemplify a coupled pore structure at interior portion 200. Due to the arrangement of the different regions in the device 100, the device 100 has what is called an “open-tight-open-tight-open” or “OTOTO” configuration. In addition, in some embodiments, the external surfaces (e.g., 600 and 604) are treated with a hydrophilic coating, such as poly vinyl alcohol (PVA) to facilitate more rapid tissue ingrowth and allow for immediate water-based liquid transport through the external portions 202 and 204. In such embodiments, the coating is thin and does not significantly significant thickness to the device 150.

Various aqueous humor flow paths can be present within the microporous material. Relatively linear flow paths may comprise regions SP1-SP2-SP3, for example, or SP1-SP2-MSP1-SP3-SP4-SP5. Although some flow paths may be relatively straight, there are also flow paths that are nonlinear. For instance, under certain conditions, at least some flow may proceed to flow through areas of increasingly less resistance such as SP1-MLP1-MLP2-LP1 or SP7-MSP2-LP2. In some examples, under certain conditions, the flow may proceed horizontally such as MLP2-LP1-SP6. As will be appreciated, the microstructure of the microporous materials may undergo modification processes to obtain certain types of flow through the microstructure. For instance, the microstructure may have relatively uniform regions across layered within the microstructure, or as shown here, have variable portions throughout the thickness of the microporous material. In some examples, the internal portion 200 defines a wall portion thickness extending between the partitions 602 (defined by SP4) and 606 (defined by SP1). The wall portion thickness can have a transition porosity that transitions between low and high porosities.

Examples of the delivery path (alternatively referred to as the flow path or passage) are shown using white dotted arrows, which indicate a general direction of the delivery path, such as into the implant interior 200 from either surface 203 or 205, and within the interior 200, flowing in a direction substantially along or parallel to the length of the implant (or in a direction substantially perpendicular to the thickness of the implant). In some examples, the direction of the delivery path inside the implant interior 200 may be substantially along the direction in which the fibrils are located within the interior 200, as shown. It is to be understood that, in some examples, either one or both the surfaces 203 and 205 may provide the inflow 36A for the excess aqueous humor, so as to direct the fluid toward the outflow surface 308 as shown in FIGS. 3B and 4B, for example.

In some examples, the device 100 includes the plurality of nodes 500 and fibrils 502 as shown in FIG. 5. The node, in some examples, includes sections with the small pore size (SP) as explained above with regard to FIG. 6, for example SP5, SP6, and SP7. The fibrils may extend between sections with small pore size, for example in LP2 located between SP6 and SP7. The device 100 is thus formed of a plurality of such nodes and fibrils that are interconnected, interlocked, or interweaved with each other, as appropriate. In some examples, the nodes have different sizes and porosities, and the fibrils may define spaces or openings having the larger pore sizes (LP), such that the pore sizes of the section defined by the fibrils are larger than the pore sizes of the nodes. In some examples, a size of the openings or spaces may be defined by a distance between neighboring nodes (e.g., an internodal distance). The pores may have a range of sizes, such as from 1 μm to 100 μm, which are evenly distributed between the solid portions. In some examples, the size (or average size) of such pores may be from about 1 μm to about 5 μm, from about 5 μm to about 10 μm, from about 10 μm to about 15 μm, from about 15 μm to about 20 μm, from about 20 μm to about 30 μm, from about 30 μm to about 40 μm, from about 40 μm to about 50 μm, from about 50 μm to about 60 μm, from about 60 μm to about 70 μm, from about 70 μm to about 80 μm, from about 80 μm to about 90 μm, from about 90 μm to about 100 μm, or any other suitable value/range therebetween or combination of ranges thereof. In some examples, the pore portions are flexible (e.g., the solid portions are made of a flexible material) to allow expansion of the pores, such that the size of the pores may vary.

In some examples, the tissue-inhibiting (or ingrowth-inhibiting) pore structure may be defined as having a minimum or average pore size of no greater than 5 μm, no greater than 4 μm, no greater than 3 μm, no greater than 2 μm, no greater than 1 μm, or any other suitable value therebetween. In some examples, the minimum or average pore size may be from 0.01 μm to 0.1 μm, from 0.1 μm to 0.3 μm, from 0.3 μm to 0.5 μm, from 0.5 μm to 0.7 μm, from 0.7 μm to 1 μm, or any other suitable value therebetween. For example, referring to FIG. 6, the tissue-inhibiting (or ingrowth-inhibiting) pore structure may include the barriers or partitions 602 and 606, where the barrier or partition 602 inhibits tissue ingrowth from the top surface 203, and the barrier or partition 606 inhibits tissue ingrowth from the bottom surface 205.

In some examples, the ingrowth-permitting pore structure may be defined as having a maximum or average pore size of 10 μm or more, 20 μm or more, 30 μm or more, 40 μm or more, 50 μm or more, 100 μm or more, or any other suitable value therebetween.

In some examples, the maximum or average pore size may be up to 200 μm, up to 300 μm, up to 500 μm, or up to 700 μm. For example, referring to FIG. 6, the ingrowth-permitting pore structure may include the first external regions 202 and 204 having the external surfaces 600 and 604 defining the top surface 203 and the bottom surface 205, respectively, having the regions marked as “LP3” and “LP4” containing the fibrils 502. It is to be understood that both the ingrowth-inhibiting pore structure and the ingrowth-permitting pore structure provide sufficient pore sizes to facilitate fluid passage through the structure (e.g., passage of aqueous humor whose general direction of flow is shown by the dotted white arrows in FIG. 6). In some embodiments, the pores are coupled to one another such that each pore fluidly communicates to one or more adjacent pores. Such a configuration stands in contrast with blind pores that begin at a surface and extend inward slightly before terminating and closed pores that are internal to a substrate and are completely encapsulated. Thus, a coupled pore structure allows fluid (e.g., the aqueous humor) to flow from one pore to another pore through the interior 200.

In some examples, the coupled pore structure may be defined as having a plurality of regions having different pore sizes, such as a first region having a minimum or average pore size of no greater than 5 μm, no greater than 4 μm, no greater than 3 μm, no greater than 2 μm, no greater than 1 μm, or any other suitable value therebetween, and a second region having a maximum or average pore size of 10 μm or more, 20 μm or more, 30 μm or more, 40 μm or more, 50 μm or more, or any other suitable value therebetween. The first region of the coupled pore structure provides the structural integrity for the device to couple together the opposing surfaces of the device (such as coupling together the opposing tissue-inhibiting or ingrowth-inhibiting pore structures, e.g., the barriers or partitions 602 and 606), and the second region of the coupled pore structure provides a region with sufficient pore sizes to facilitate fluid passage through the structure (e.g., passage of aqueous humor whose general direction of flow is shown by the dotted white arrows in FIG. 6). For example, referring to FIG. 6, the first region may include the support sections 608, and the second region may be the region between neighboring support sections 608, such as in the regions marked as “LP1” or “LP2” containing the fibrils 502.

There are numerous different testing or measurement processes which can be performed on the pore structures of the implantable devices as disclosed herein, for example including but not limited to: Gurley test, Sheffield test, Bendtsen test, mercury intrusion porosimetry, gravimetric method, liquid displacement method, optical microscopy, image analysis, and visual inspection. Each of the processes is explained herein.

“Gurley” is a measurement of the resistance of a porous sample to airflow under a given pressure drop. Gurley is defined as the time in seconds that it takes for 100 cm³ of air to pass through one square inch of a sample material when a constant pressure of 4.88 inches of water (0.177 psi) is applied. A higher Gurley number indicates lower air permeability or greater resistance to airflow under a given pressure drop. Gurley is reported in units of seconds or (s/(100 cm³*in²)) at 0.177 psi. The Gurley test measures the Gurley number of a material by measuring the time required for a certain volume of air to flow through a material under a constant pressure difference. The Gurley test, also referred to as the Gurley air permeability test, is widely used for measuring the porosity of paper, textiles, and filters. The test may be performed using any suitable flowmeter, air resistance tester, and/or densometer, such as the Model 4340 Automatic Densometer & Smoothness Tester as manufactured by Gurley Precision Instruments, Inc. (Troy, New York, U.S.A.), for example. An example of a standard test method for such measurements may be the TAPPI T460 Gurley air resistance test or the ISO 5636-5 Porosity test, as known in the art.

In some examples, the permeability may be calculated using the following equation:

$$Permeability = \frac{Q η h}{Ftp} \qquad ( Equation 1 )$$

where Q is the volume of fluid that passed through the sample of a period of time (t), rn is the viscosity of the fluid, h is the thickness of the sample, F is the cross-sectional area of the sample perpendicular to the fluid flow, and p is the applied head pressure. After calculating the permeability, the pore radius of the sample may be calculated using the following equation:

$$Pore Radius = \frac{0.5093}{{( Permeability )}^{- \frac{1}{2}}} \qquad ( Equation 2 )$$

The Sheffield test measures the flow rate of air through a material at different pressure differences. The Sheffield test is also typically used for measuring the porosity of paper, textiles, and filters. The test may be performed by placing the sample material (specimen) into rubber clamping rings. Compressed air is passed through a flow measuring device and then directed to the specimen test area. The air that passes through the specimen escapes to the atmosphere through holes in the downstream clamping plate, and the air flow is measured to determine the air permeance of the specimen.

The Bendtsen test measures the flow rate of air through a material at a constant pressure difference and a constant area. The Bendtsen test is typically used for measuring the roughness and porosity of paper and board. The Bendtsen test is based upon a leak principle. A sample material or specimen is clamped between a flat glass plate and a circular metal head. Air is then forced through the sample, and the rate of airflow between the sample and the head is measured in mL/minute. It is a commonly used testing technique with an adjustable testing time and water pressure. Examples of a device which may be used to perform the Sheffield test and/or the Bendtsen test may include, for example, the 58-27 Bendtsen Tester as manufactured by Büchel B.V. (Capelle aan den Ijssel, the Netherlands). An example of a standard test method for such measurements may be the ISO 5636-3 Bendtsen Method test, as known in the art.

The mercury intrusion porosimetry (MIP) test or technique measures the volume of mercury that intrudes into the pores of a material under increasing pressure. MIP is typically used for measuring the porosity and pore size distribution of materials such as ceramics, metals, and composites. The MIP technique is based upon the pressure-dependent intrusion of mercury as a non-wetting liquid into a porous material. Using Washburn's equation, the corresponding pore size can be calculated based upon the applied pressure. Examples of a device which may be used to perform the Sheffield test and/or the Bendtsen test may include, for example, the BELPORE porosimeter series such as BELPORE LP, MP, and HP as manufactured by MicrotracBEL Corp (Osaka, Japan). An example of a standard test method for such measurements may be the ISO 15901-1:2016 Mercury Porosimetry test and/or ISO 15901-2:2022 Analysis of Nanopores by Gas Adsorption, as known in the art. A minimum, maximum, and/or average pore size can be measured or calculated from data collected from the above-identified techniques using known mathematical and statistical methods.

The gravimetric method uses a bulk density and a true density of a porous material sample to determine its total porosity. A “bulk density” may be calculated by simply dividing the mass of the porous sample by its total volume (e.g., total volume of the porous sample being the volume of solid content added to the volume of void content). A “true density” may be determined using a helium pycnometer (or any other suitable gas pycnometer as known in the art) which measures the volume of only the solid content in the porous sample using Boyle's Law which is known as “true volume.” Since the mass of the sample is known, the true density may be obtained by dividing the mass of the sample by its true volume. Using the above metrics, the sample's porosity may be calculated to define the measurement of the void content in the porous material, where a “percentage porosity” may be calculated using the below equation:

$$\% Porosity = ( 1 - \frac{B}{T} ) * 100 \qquad ( Equation 3 )$$

where B is the bulk density and T is the true density of the porous material.

The liquid displacement method involves using a displacement liquid that is not a solvent of the polymers (e.g., ethanol) of a porous material sample and is capable of penetrating into the pores easily but do not cause size shrinkage or swelling to the material being tested. The sample may be placed in a cylinder with a known volume of the displacement liquid and a series of evacuation-repressurization cycles may be performed to force the liquid into the pores, as an indirect way of measuring porosity. An “open porosity” of the material may be calculated using the following equation:

$$Porosity = \frac{{V}_{1} - {V}_{3}}{{V}_{2} - {V}_{3}} \qquad ( Equation 4 )$$

where V1 is the known volume of liquid that is used to submerge the sample (but not a solvent for the material of the sample), V2 is the volume of the liquid and liquid-impregnated sample, and V3 is the remaining liquid volume when the liquid-impregnated sample is removed.

The optical microscopy test or technique uses a microscope to examine the material at a high magnification and identify the pores or voids in the material. The porosity can be estimated by counting the number of pores or measuring the area of the pores relative to the total area of the material. The estimation and/or the counting may be performed manually or with the use of a computer running an algorithm or software that is programmed to perform such task. In some examples, the algorithm or software may implement machine learning or artificial intelligence technology to automatically detect and identify pores, as suitable.

The image analysis test or technique uses a digital image of the material, such as an image obtained using scanning electron microscopy (SEM), microcomputed tomography (Micro-CT) imaging, or a 3D model, for example, and a software program to analyze the image and calculate the porosity. The image can be obtained from various sources, such as optical microscopy, scanning electron microscopy, or computed tomography. The software can use different algorithms, such as thresholding, segmentation, or edge detection, to distinguish the pores from the solid phase and measure the porosity. In some examples, the software may implement machine learning or artificial intelligence technology to automatically analyze the digital image, as suitable.

The visual inspection test or technique uses a human's naked eyes or a low-power magnification device to manually observe the material and estimate the porosity based on its texture, color, or transparency. This method is subjective and qualitative, and may be more suitable for materials with simpler or larger pores.

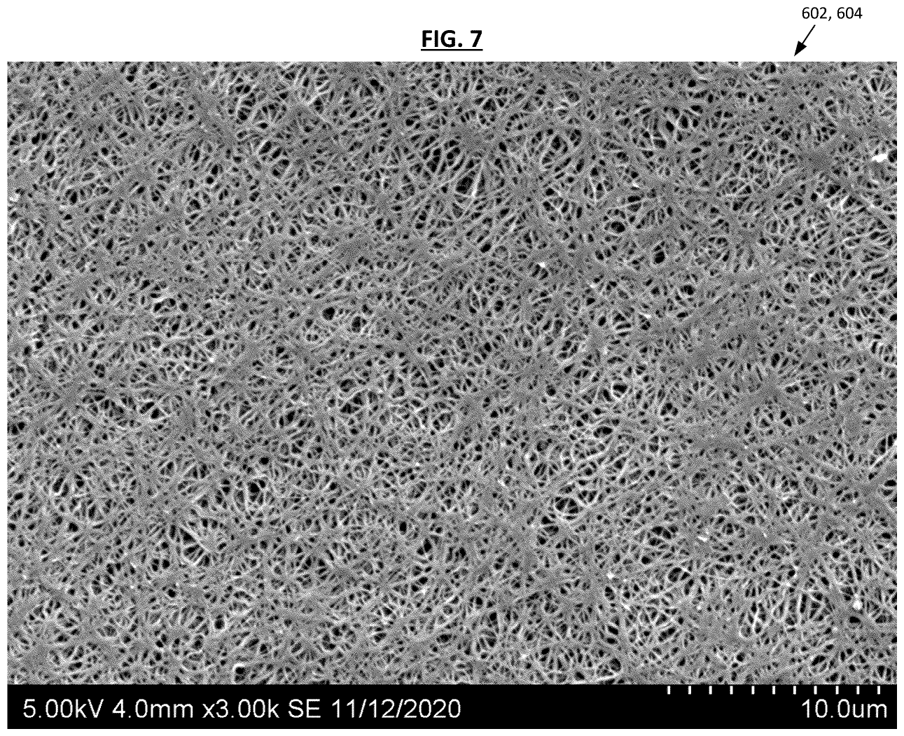

FIG. 7 shows an example of a surface of the first region of barrier or partition 602 or the second region of barrier or partition 604, as viewed from above. The figure shows a microscopic view of a microporous material of the barrier or partition according to some embodiments. A scale is displayed at the bottom of FIG. 7 is: “5.00 kV 4.0 mm×3.00 k SE 11/12/2020,” showing the distance between two consecutive lines to represent 1.0 μm. The barrier(s) or partition(s) 602 and/or 604 may have a small pore size (SP) or a porosity that is sufficiently small to prevent or inhibit tissue ingrowth therein. The barrier or partition may be formed as a single continuous node, where the node is made or formed of an unexpanded or compressed material such as PE or PTFE, for example. For example, a node may be defined by the initial state or density of the material in its unexpanded form (also referred to herein as an unexpanded material, or a compressed material, such as PE or PTFE), while the fibrils may be formed by applying an expansion force in one or more directions to the unexpanded material so as to pull apart the unexpanded material in one or more directions to define a plurality of portions with lesser density than the initial state of the material. For example, the fibrils may be defined as the expanded portions (e.g., portions having physical properties similar to expanded materials such as ePE or ePTFE), and the nodes may be defined as the unexpanded (or lesser expanded) portions of the material, as compared to the expanded portions (e.g., with the unexpanded portions having physical properties similar to unexpanded or compressed materials such as PE or PTFE). In some examples, the nodes may be defined as having higher density and smaller pore sizes than the remaining portion(s) having the fibrils, and the remaining portion(s) having the fibrils may have lower density and larger pore sizes (where a pore may be at least partially defined by interstitial spaces located between neighboring fibrils) as compared to the nodes. As shown, the surface has a plurality of fibrils that are interconnected and concentrated in the way that forms a layered structure. In a layered structure, the fibrils forming an upper region may include a plurality of openings, and similarly the fibrils forming a lower region underneath the upper region may also include a plurality of openings. However, because the openings in each region are located in different locations, the difference in the displacement of the openings prevents, inhibits, or reduces the amount of tissue ingrowth from the top region through to the bottom region, thereby causing the bottommost region to remain substantially free of tissue ingrowth, thereby providing a tissue-inhibiting pore structure.

Using the device or implant 100 as disclosed herein, a surgeon or medical practitioner may perform drainage of excessive aqueous humor from a human eye 10 to treat glaucoma in conjunction with the DS surgical procedure defining the scleral space 32, as disclosed herein. In some examples, the drainage involves directing the inflow 36A from an exposed Schlemm's canal internal wall 34 into the interior 200 of the scleral space implant 100 disposed in the scleral space. In some examples, the drainage also involves inhibiting an ingress of tissue ingrowth from the scleral space 32 surrounding the implant 100 into the implant interior 200. Thereafter, in some examples, the drainage involves channeling the inflow of the fluid (excess aqueous humor) through the implant interior 200 toward the outflow or exit 36B, 36C that communicates with an exterior of the eye 10. In some examples, the route (or alternatively referred to as a flow path or passage) of the fluid within the implant 100 may be located along any one or more of the arrows as shown in FIGS. 1, 2E, 3B, 4B, 5, and/or 6. In some examples, the peripheral edge of the scleral space implant may be trimmed to conform to the scleral space. In some examples, the superficial scleral flap may be supported in a planar orientation.

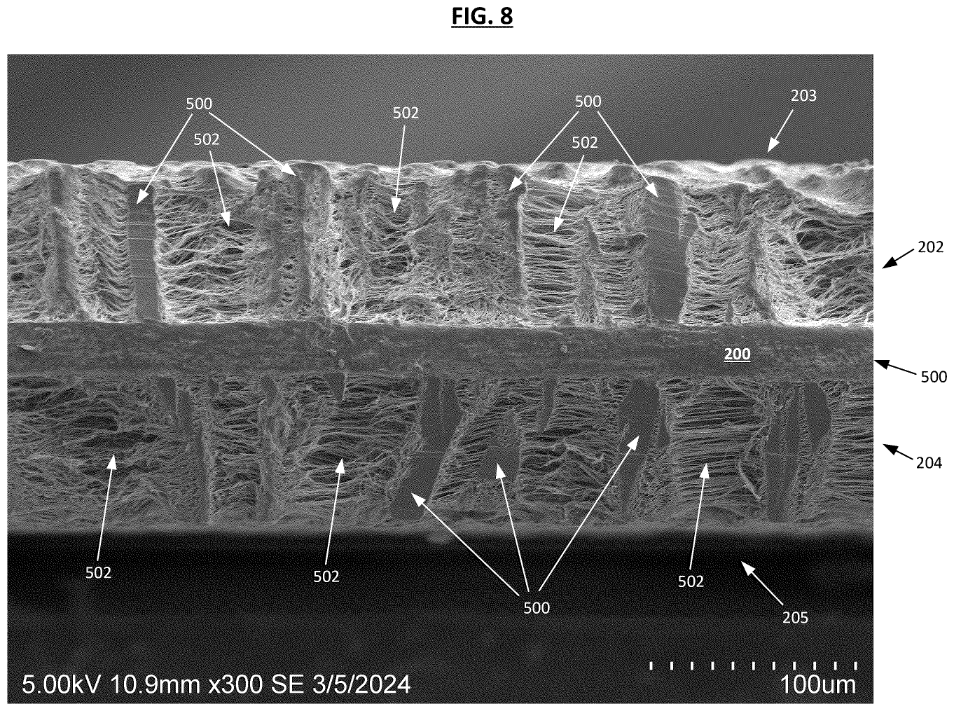

FIG. 8 is an SEM image of a cross sectional view of an implantable device according to embodiments disclosed herein. A scale is displayed at the bottom of FIG. 8 is: “5.00 kV 10.9 mm×300 SE 3/5/2024,” showing the distance between two consecutive lines to represent 10 μm. Similar to the embodiment shown in FIG. 2F, the SEM image shows a substrate with three (3) regions: a first external region 202, a second external region 204, and an internal region 200 disposed between the external regions 202 and 204, where the internal region or implant interior 200 is composed of a material or substrate that includes a tissue-inhibiting pore structure (which may be a single continuous node 500 with additional nodes extending from each side thereof, as shown), whereas the external regions 202 and 204 are composed of a material or substrate that includes a ingrowth-permitting pore structure (with may include an interconnected network of nodes 500 and fibrils 502 with fibrils 502 extending between neighboring nodes 500). In some examples, the fibrils and the nodes may be formed using the same material. Therefore, the top surface 203 and the bottom surface 205 may at least partially include ingrowth-permitting pore structures, with the pores being at least partially defined by the spacings (such as interstitial spaces) between nodes 500 and fibrils 502. As such, in some examples, the top surface 203 and the bottom surface 205 may have pores with a maximum or an average pore size from 1 μm to 5 μm, from 5 μm to 10 μm, from 10 μm to 20 μm, from 20 μm to 30 μm, from 30 μm to 40 μm, from 40 μm to 50 μm, from 50 μm to 60 μm, from 60 μm to 70 μm, from 70 μm to 80 μm, from 80 μm to 90 μm, from 90 μm to 100 μm, or any other suitable value/range therebetween or combination of ranges thereof, as part of the ingrowth-permitting pore structures. In some examples, the external regions 202 and 204 may be formed entirely of tissue-inhibiting pore structures, such as one or more nodes 500, such that the internal region 200 and the external regions 202 and 204 may have a similar pore structure. In some examples, the internal region 200 and the external regions 202 and 204 may be unitary or formed as a single continuous piece of material, such that the regions 200, 202, and 204 may be considered a single continuous region having the material shown in region 200 extending the distance between the surfaces 203 and 205. In another embodiment, the region 200 may have sufficient thickness to extend as a monolithic region over the distance between the surfaces 203 and 205 such that the region 200 defines the surfaces 203 and 205.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A scleral space implant for implantation in a scleral space created during a deep sclerectomy surgical procedure performed on a human eye, the deep sclerectomy surgical procedure involving a removal of eye tissue to define the scleral space between a superficial scleral flap and a Schlemm's canal internal wall of the eye, the Schlemm's canal internal wall defining an inflow of an aqueous humor into the scleral space, the scleral space implant comprising:
a singular porous substrate having a top surface and an opposing bottom surface defining an implant interior and a consistent implant thickness disposed uniformly between the opposing top and bottom surfaces, the substrate further having an implant rigidity that maintains the implant thickness when the scleral space implant is disposed in the scleral space,
wherein the top and bottom surfaces of the substrate each have a tissue-inhibiting pore structure that inhibits an ingress of ingrowing tissue into the implant interior while permitting the inflow of the aqueous humor into the implant interior, and
wherein the implant interior has a coupled pore structure that facilitates a passage of the aqueous humor inflow through the implant interior.

2. The implant of claim 1, wherein the singular porous substrate further includes at least one exterior region having an ingrowth-permitting pore structure that permits the ingress of ingrowing tissue into the at least one exterior region.

3. The implant of claim 2, wherein the singular porous substrate has an open-tight-open-tight-open configuration.

4. The implant of claim 1, wherein the top and bottom surfaces each define peripheral edges that mate to fully enclose the implant interior in at least one cross-sectional plane passing through the implant.

5. The implant of claim 1, wherein the singular porous substrate is trimmable to define an implant profile conforming to the scleral space.

6. The implant of claim 5, wherein the implant, after being trimmed and implanted, is disposed entirely beneath the superficial scleral flap.

7. The implant of claim 1, wherein the top surface defines a scleral flap plane for receiving the superficial scleral flap and maintaining the superficial scleral flap in a planar orientation.

8. The implant of claim 1, wherein the passage extends from the bottom surface adjacent to the Schlemm's canal internal wall through the implant interior to an outflow surface of the implant facilitating a removal of the aqueous humor from the scleral space.

9. The implant of claim 1, wherein the singular porous substrate includes a tail portion extending from the scleral space.

10. The implant of claim 9, wherein the outflow surface is disposed at the tail portion.

11. The implant of claim 10, wherein the tail portion is trimmable to be flush with an outer surface of the superficial scleral flap.

12. The implant of claim 1, wherein the singular porous substrate includes polyethylene (PE), expanded polyethylene (ePE), expanded polytetrafluoroethylene (ePTFE), or silicone.

13. The implant of claim 1, wherein the deep sclerectomy surgical procedure does not involve the use of an antimetabolite.

14. A scleral space implant for implantation in a scleral space created during a deep sclerectomy surgical procedure performed on a human eye, the deep sclerectomy surgical procedure involving a removal of eye tissue to define the scleral space between a superficial scleral flap and a Schlemm's canal internal wall of the eye, the Schlemm's canal internal wall defining an inflow of an aqueous humor into the scleral space, the scleral space implant comprising:
a singular porous substrate having a top surface and an opposing bottom surface defining an implant interior and a consistent implant thickness disposed uniformly between the opposing top and bottom surfaces, the substrate further having an implant rigidity that maintains the implant thickness when the scleral space implant is disposed in the scleral space,
wherein the singular porous substrate includes a tissue-inhibiting pore structure extending through a majority of the implant thickness, the tissue-inhibiting pore structure inhibiting an ingress of ingrowing tissue into the implant interior while permitting the inflow of the aqueous humor into the implant interior.

15. The implant of claim 14, wherein the singular porous substrate further includes at least one exterior layer having an ingrowth-permitting pore structure that permits the ingress of ingrowing tissue into the at least one exterior layer such that the singular porous substrate has an open-tight-open-tight-open configuration.

16. The implant of claim 14, wherein the singular porous substrate includes polyethylene (PE), expanded polyethylene (ePE), expanded polytetrafluoroethylene (ePTFE), or silicone.

17. The implant of claim 14, wherein the singular porous substrate is trimmable to define an implant profile conforming to the scleral space.

18. The implant of claim 14, wherein the singular porous substrate includes a tail portion extending from the scleral space.

19. The implant of claim 14, wherein the deep sclerectomy surgical procedure does not involve the use of an antimetabolite.

20. The implant of claim 14, wherein the implant interior has a coupled pore structure that facilitates a passage of the aqueous humor inflow through the implant interior.
